(19) United States
(12) Patent Application Publication
(10) Pub. No.: US 2003/0190652 A1
(43) Pub. Date: Oct. 9, 2003

(54) Methods of validating SNPs and compiling libraries of assays

(76) Inventors: Francisco M. De La Vega, San Mateo, CA (US); Janet S. Ziegle, Berkeley, CA (US); Hadar I. Avi-Itzhak, Foster City, CA (US); Charles R. Scafe, San Francisco, CA (US); Eugene G. Spier, Palo Alto, CA (US); Yu N. Wang, Pittsburgh, PA (US)

Correspondence Address: KILYK & BOWERSOX, P.L.L.C., 3603 CHAIN BRIDGE ROAD, SUITE E, FAIRFAX, VA, 22030, US

(21) Appl. No.: 10/335,707

(22) Filed: Jan. 2, 2003

Related U.S. Application Data
(60) Provisional application No. 60/352,039, filed on Jan. 25, 2002.
(60) Provisional application No. 60/352,356, filed on Jan. 28, 2002.
(60) Provisional application No. 60/369,127, filed on Apr. 1, 2002.
(60) Provisional application No. 60/369,657, filed on Apr. 3, 2002.
(60) Provisional application No. 60/370,921, filed on Apr. 9, 2002.
(60) Provisional application No. 60/376,171, filed on Apr. 26, 2002.
(60) Provisional application No. 60/380,057, filed on May 6, 2002.
(60) Provisional application No. 60/383,627, filed on May 28, 2002.
(60) Provisional application No. 60/390,708, filed on Jun. 21, 2002.
(60) Provisional application No. 60/394,115, filed on Jul. 5, 2002.
(60) Provisional application No. 60/399,860, filed on Jul. 31, 2002.

(51) Int. Cl. C12Q 1/68; G06F 19/00; G01N 33/48; G01N 33/50

(52) U.S. Cl. 435/6; 702/20

(57) ABSTRACT

Libraries of assays and methods of compiling the libraries are provided. The assays can identify Single Nucleotide Polymorphisms (SNPs). Methods of validating SNPs are provided. Methods of constructing linkage disequilibrium maps using sets or subsets of SNPs are also provided.

### CROSS REFERENCE TO RELATED APPLICATIONS

This application claims the benefit under 35 U.S.C. § 119(e) of prior U.S. Provisional Patent Applications Nos. 60/352,039, filed Jan. 25, 2002; 60/352,356, filed Jan. 28, 2002; 60/369,127, filed Apr. 1, 2002; 60/369,657, filed Apr. 3, 2002; 60/370,921, filed Apr. 9, 2002; 60/376,171, filed Apr. 26, 2002; 60/380,057, filed May 6, 2002; 60/383,627, filed May 28, 2002; 60/383,954, filed May 29, 2002; 60/390,708, filed Jun. 21, 2002; 60/394,115, filed Jul. 5, 2002; and 60/399,860, filed Jul. 31, 2002; U.S. Non-Provisional Patent Application No. ______ by De La Vega et al., entitled “Single-Tube, Ready-To-Use Assay Kits And Methods Using Same” [Atty. Docket No. 4797 (5010-022-13)], filed concurrently herewith; and U.S. Non-Provisional Patent Application No. ______ by Koehler et al entitled “Methods For Placing, Accepting and Filling Orders For Products And Services” (Atty. Docket No. 9692-000017/US), filed concurrently herewith; all of which are incorporated herein in their entireties by reference.

### BACKGROUND

Assays can include probes and/or primers that hybridize with a target nucleic acid sequence. These probes and primers can be useful for visualizing or amplifying the target nucleic acid sequence. The target nucleic acid sequence can have a Single Nucleotide Polymorphism (SNP) contained therein. A relative indication of concentration of the target nucleic acid sequence can be obtained based on relative fluorescence of the probes.

Single nucleotide polymorphisms (SNPs) are an abundant form of genetic variation. These single nucleotide changes are found approximately every 500 bp in the human genome. Almost all SNPs are bi-allelic, that is, only two different alleles exist. Typically, one allele is present in the majority of the chromosomes of a population, and the alternative variant, that is, the minor allele, is present with less frequency. Only alleles that are present at a frequency greater than 1% are considered polymorphisms.

SNPs are promising tools for mapping susceptibility mutations that contribute to complex diseases. Although most SNPs are neutral and do not affect phenotype, they can be used as surrogate markers for positional cloning of genetic loci, because of the allelic association, known as linkage disequilibrium (LD), that can be shared by groups of adjacent SNPs. LD is eroded by gene conversion and recombination, and the amount of LD depends on the age of the mutations and on the demographic history of the population. The extent of LD across a genomic region dictates the density of SNP markers necessary to ensure association between a marker and the causative allele sought.

Early attempts to model the extent of LD on theoretical grounds predicted very short regions of LD, extending only a few kilobases (Kb). However, empirical surveys reported average LD distances between 5 Kb and 60 Kb, with the upper range extending up to hundreds of Kb.

Previous efforts for typing the specific SNP alleles present in a DNA sample produce unphased genotypes (i.e., the alleles detected cannot be assigned to either the maternal or the paternal chromosome). Although there are a few cumbersome methods to directly determine haplotypes, previous algorithms are widely used to infer the haplotypes from genotypes using maximum-likelihood or Bayesian principles.

### SUMMARY

According to various embodiments, methods are provided for SNP validation that take into consideration a number of findings and statistics. Studies have reported a discontinuous structure in the patterns of LD across a set of regions sampled from the human genome, where long stretches of strong LD are punctuated by recombination hot-spots. These LD “blocks” show little evidence of historical recombination. According to various embodiments, these results are deconvoluted to predict that a reduced set of contiguous chromosomal segments, or haplotypes, exist in specific populations. For example, for a block spanning tens of Kbs for which 10 SNPs exist, instead of the 2¹⁰ theoretically possible haplotypes, it has been found that 95% of the haplotype diversity is made up on only 4 to 6 so-called common haplotypes.

It is noteworthy that these LD block patterns change depending on the population sampled because of historical differences; for example, populations that have experienced bottlenecks (e.g., Caucasians) show longer LD blocks and less evidence of historical recombination events, than other populations. The haplotype diversity in a given population is typically constant in a given region irrespective of the number of SNPs sampled; therefore typing an arbitrarily large number of SNPs within a LD block is unnecessary. Selecting the minimum subset of SNPs within LD blocks, or any other discrete genetic locus, that enable discrimination of the common haplotypes present in a block without loss of information can be used to validate SNPs and/or to compile a concise library of assays useful for genetic analysis.

According to various embodiments, a method of compiling a library of polynucleotide data sets is provided. The data sets can correspond to polynucleotides that each can function as (A) a primer for producing a nucleic acid sequence that is complementary to at least one target nucleic acid sequence including a target SNP, (B) a probe for rendering detectable the at least one target nucleic acid sequence including a target SNP, or (C) both (A) and (B). The method can include the step of selecting for the library polynucleotide data sets that each correspond to a respective polynucleotide that contains a sequence that is complementary to a respective first allele included in each of the at least one target nucleic acid sequences, if, under a set of reaction conditions a number of parameters are met by each polynucleotide corresponding to the data sets included in the library. The parameters can include: (1) the respective polynucleotide has a background signal value less than or equal to a first defined value, where the background signal value is a first normalized ratio of a fluorescence intensity of the respective polynucleotide reacted with first assay reactants in the absence of the target nucleic acid sequence, and under first conditions of fluorescence excitation, to a dye fluorescence intensity of a passive-reference dye under the first conditions; (2) the respective polynucleotide has a signal generation value of greater than or equal to a second defined value, wherein the signal generation value is the difference between (i) a second normalized ratio of the fluorescence intensity of the respective polynucleotide reacted with the first assay reactants in the presence of the target nucleic acid sequence, to the dye fluorescence intensity and (ii) the background signal value; (3) the respective polynucleotide has a specificity value of less than or equal to a third defined value, wherein the specificity value is the difference between (i) a third normalized ratio of the fluorescence intensity of the respective polynucleotide reacted with second assay reactants that contain a second allele included in the at least one target nucleic acid sequence to the dye fluorescence intensity, wherein the second allele differs from the first allele, and (ii) the background signal value; (4) at least one individual from a population of individuals has a genotype identifiable under the first conditions, that results from reacting the respective polynucleotide with the first assay reactants and in the presence of the target nucleic acid sequence, wherein the population includes at least one individual that has the identifiable genotype and at least one individual that does not have the identifiable genotype; and (5) at least one individual from the population has an identifiable minor allele of the identifiable genotype, under the first conditions that results from reacting the respective polynucleotide with the first assay reactants in the presence of the target nucleic acid sequence, wherein the population includes at least one individual that has the identifiable minor allele, and at least one individual that does not have the identifiable minor allele.

According to various embodiments, a method of compiling a library of polynucleotide data sets is provided. The data sets can correspond to polynucleotides that each can function as (A) a primer for producing a nucleic acid sequence that is complementary to at least one target nucleic acid sequence including a target SNP, (B) a probe for rendering detectable the at least one target nucleic acid sequence including a target SNP, or (C) both (A) and (B). The method can include the step of determining a background signal value by calculating a first normalized ratio of a fluorescence intensity of a respective polynucleotide that contains a sequence that is complementary to a first allele included in the at least one target nucleic acid sequence, reacted with first assay reactants in the absence of the target nucleic acid sequence, and under first conditions of fluorescence excitation, to a dye fluorescence intensity of a passive-reference dye under the first conditions. The method can include the step of comparing a difference between (i) a second normalized ratio of the fluorescence intensity of the respective polynucleotide reacted with the first assay reactants in the presence of the target nucleic acid sequence, to the dye fluorescence intensity, and (ii) the background signal value. The method can include the step of comparing a difference between (i) a third normalized ratio of the fluorescence intensity of the respective polynucleotide reacted with second assay reactants that contain a second allele included in the at least one target nucleic acid sequence to the dye fluorescence intensity, wherein the second allele differs from the first allele, and (ii) the background signal value. The method can include the step of determining whether at least one individual from a population of individuals has a genotype identifiable under the first conditions that results from reacting the respective polynucleotide with the first assay reactants and in the presence of the target nucleic acid sequence, wherein the population includes at least one individual that has the identifiable genotype and at least one individual that does not have the identifiable genotype. The method can include the step of determining whether at least one individual from the population has an identifiable minor allele of the identifiable genotype, under the first conditions that results from reacting the respective polynucleotide with the first assay reactants in the presence of the target nucleic acid sequence. Various combinations of the herein described method steps and/or parameters can be used.

According to various embodiments, a method of confirming the existence of a SNP is provided. The method can include the step of identifying a location corresponding to a possible SNP in a polynucleotide in a first collection of data sets containing information on genomic deoxyribonucleic acid (DNA) samples in the form of data sets corresponding to polynucleotides. The method can include the step of confirming the existence of the SNP if at least one condition is met. A condition can be met if a second collection of data sets containing information on genomic deoxyribonucleic acid (DNA) samples contains information that identifies the location as containing the possible SNP. A condition can be met, for example, if at least two data sets from the first collection of data sets contain information corresponding to a minor allele of the possible SNP at the location, wherein the at least two data sets represent genomic deoxyribonucleic acid (DNA) samples obtained from two independent sources. A condition can be met if a data set that corresponds to a consensus sequence of genomic deoxyribonucleic acid (DNA) samples that contains the minor allele of the possible SNP in a third collection of data sets. The source of the consensus sequence of genomic deoxyribonucleic acid (DNA) samples, and the sources of the genomic deoxyribonucleic acid (DNA) samples from the first collection of data sets, can be independent.

According to various embodiments, a library is provided that contains data corresponding to respective oligonucleotides that can function as assays to detect Single Nucleotide Polymorphisms (SNPs). The library can have a number of data sets corresponding to not more than a sufficient number of oligonucleotides necessary to provide a collection of assays that provides a maximum statistical loss of a defined percentage of haplotype diversity across a human genome.

According to various embodiments, an algorithm to select the minimal subset of SNPs required for capturing the diversity of haplotype blocks or other genetic loci is provided. The algorithm can be used to quickly select the minimum SNP subset with no loss of haplotype information. In addition, the algorithm can be used in a more aggressive mode to further reduce the original SNP set, with minimal loss of information.

### BRIEF DESCRIPTION OF THE DRAWINGS

FIGS. 1a-b are graphs of SNPs per LD block v. minimum information SNP subset for African-American and Caucasian populations, respectively;

FIGS. 2a-2e are schematic diagrams of quenchable dyes that can be part of a mixture of reagents provided and/or used according to various embodiments;

FIG. 3 is a workflow diagram according to various embodiments;

FIG. 4 is a graph showing visualized assay results, according to various embodiments;

FIG. 5 is a flowchart showing an algorithm according to various embodiments; and

FIG. 6 is a flowchart showing an algorithm according to various embodiments.

### DESCRIPTION OF VARIOUS EMBODIMENTS

According to various embodiments, nucleic acid analogs can be used in addition to or instead of nucleic acids. Examples of nucleic acid analogs can include the family of peptide nucleic acids (PNA), wherein the sugar/phosphate backbone of DNA or RNA has been replaced with acyclic, achiral, and neutral polyamide linkages. For example, a probe or primer can have a PNA polymer instead of a DNA polymer. The 2-aminoethylglycine polyamide linkage with nucleobases attached to the linkage through an amide bond can be used as a PNA and shown to possess exceptional hybridization specificity and affinity. An example of a PNA is as shown below in a partial structure with a carboxyl-terminal amide:

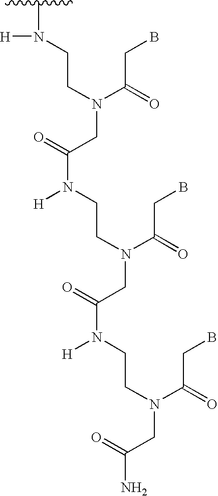

“Nucleobase” as used herein means any nitrogen-containing heterocyclic moiety capable of forming Watson-Crick hydrogen bonds in pairing with a complementary nucleobase or nucleobase analog, e.g. a purine, a 7-deazapurine, or a pyrimidine. Typical nucleobases are the naturally occurring nucleobases such as, for example, adenine, guanine, cytosine, uracil, thymine, and analogs of the naturally occurring nucleobases, e.g. 7-deazaadenine, 7-deazaguanine, 7-deaza-8-azaguanine, 7-deaza-8-azaadenine, inosine, nebularine, nitropyrrole, nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-azapurine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG”, and ethenoadenine.

“Nucleoside” as used herein refers to a compound consisting of a nucleobase linked to the C-1′ carbon of a sugar, such as, for example, ribose, arabinose, xylose, and pyranose, in the natural β or the α anomeric configuration. The sugar can be substituted or unsubstituted. Substituted ribose sugars can include, but are not limited to, those riboses having one or more of the carbon atoms, for example, the 2′-carbon atom, substituted with one or more of the same or different Cl, F, -R, -OR, -NR₂ or halogen groups, where each R is independently H, C₁-C₆ alkyl or C₅-C₁₄ aryl. Ribose examples can include ribose, 2′-deoxyribose, 2′, 3′-dideoxyribose, 2′-haloribose, 2′-fluororibose, 2′-chlororibose, and 2′-alkylribose, e.g. 2′-O-methyl, 4′-α-anomeric nucleotides, 1′-α-anomeric nucleotides, 2′-4′- and 3′-4′-linked and other “locked” or “LNA”, bicyclic sugar modifications. Exemplary LNA sugar analogs within a polynucleotide can include the following structures:

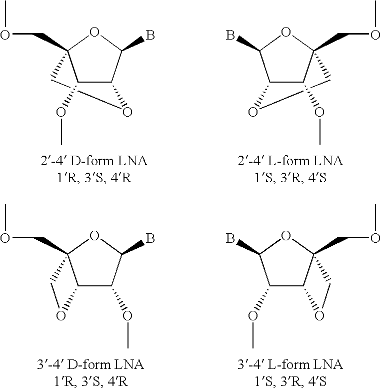

where B is any nucleobase.

Sugars can have modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides can have the natural D configurational isomer (D-form) or the L configurational isomer (L-form). When the nucleobase is a purine, e.g. adenine or guanine, the ribose sugar is attached to the N⁹-position of the nucleobase. When the nucleobase is a pyrimidine, e.g. cytosine, uracil, or thymine, the pentose sugar is attached to the N¹-position of the nucleobase.

“Nucleotide” as used herein refers to a phosphate ester of a nucleoside and can be in the form of a monomer unit or within a nucleic acid. “Nucleotide 5′-triphosphate” as used herein refers to a nucleotide with a triphosphate ester group at the 5′ position, and can be denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates.

As used herein, the terms “polynucleotide” and “oligonucleotide” mean single-stranded and double-stranded polymers of, for example, nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides can have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of internucleotide, nucleobase and sugar analogs. For example, a polynucleotide or oligonucleotide can be a PNA polymer. Polynucleotides can range in size from a few monomeric units, e.g. 5-40 when they are more commonly frequently referred to in the art as oligonucleotides, to several thousands of monomeric nucleotide units. Unless otherwise denoted, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted.

“Internucleotide analog” as used herein means a phosphate ester analog or a non-phosphate analog of a polynucleotide. Phosphate ester analogs can include: (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Non-phosphate analogs can include compounds wherein the sugar/phosphate moieties are replaced by an amide linkage, such as a 2-aminoethylglycine unit, commonly referred to as PNA.

“Heterozygous” as used herein means both members of a pair of alleles of a gene are present in a sample obtained from a single source, wherein a gene can have two alleles due to, for example, the fusion of two dissimilar gametes with respect to the gene.

“Heterozygous assay” as used herein means an assay adapted to identify the allelic state of a gene having one or both members of a pair of alleles.

“Homozygous” as used herein means one member of a pair of alleles is present in a sample obtained from a single source, wherein a gene can have one allele due to, for example, the fusion of two identical gametes with respect to the gene.

“Homozygous assay” as used herein means an assay adapted to identify only one of two possible allelic states of a gene having one or both members of a pair of alleles.

“Lossy” as used herein means the loss of haplotype diversity in a linkage disequilibrium block.

“Lossless” as used herein means that there is no loss of haplotype diversity in a linkage disequilibrium block.

According to various embodiments, a library of assays can be provided. The library of assays can have from about 100,000 to about 500,000 polynucleotides, for example, about 150,000 to about 250,000 polynucleotides. According to various embodiments, a library of data sets can be provided. The library of data sets can have from about 100,000 to about 500,000 data sets, for example, about 150,000 to about 250,000 data sets.

According to various embodiments, an algorithm is provided that can select a minimum subset of SNPs without loss of haplotype information, or an even smaller subset with some acceptable loss of information. In an example, a SNP set was reduced by 18% for an African American population and by 32% for a Caucasian population with no loss of haplotype distribution information. The algorithm can produce optimal results in a reasonable time. The algorithm can allow for the real-time calculation of minimum SNP subsets for haplotype blocks.

According to various embodiments, an algorithm to select the minimal subset of SNPs required for capturing the diversity of haplotype blocks or other genetic loci is provided. The algorithm can be used to quickly select the minimum SNP subset with no loss of haplotype information. In addition, the algorithm can be used in a more aggressive mode to further reduce the original SNP set, with minimal loss of information.

When SNPs are initially selected for typing, often not much is known about the existence or location of LD blocks, or about the number and relative frequencies of haplotypes within the blocks. It is therefore typical in previous efforts to “over-sample” the chromosomal region, (i.e., select SNPs as densely as one's budget permits). Since there may be large costs associated with detecting the genotype for each SNP, it can be practical to minimize the number of SNPs used in a study. When beginning with a population sample large enough to allow for accurate inference of the haplotype distributions, various embodiments can reduce the set of SNPs to the minimum number required for adequate coverage with no loss of haplotype information. Furthermore, various embodiments can be used to eliminate additional SNPs while minimizing loss of haplotype information.

According to various embodiments, family relationships of the DNA donors, if available, can be used to increase haplotype inference accuracy. In the absence of family information, the Expectation-Maximization algorithm introduced by Excoffier and Slatkin can be accurate, especially in regions of low diversity. According to various embodiments, the analysis of haplotype distributions in genetic studies aimed to find susceptibility mutations in case-control populations can be useful in finding associations. Therefore, haplotype interference can be used in disease and pharmacogenomic studies.

According to various embodiments, given a block containing N SNPs and M haplotypes, a probability vector P of length M can be defined where Pᵢ is the relative frequency of the i^{th} haplotype. According to various embodiments, A, a haplotype/SNP allele state matrix of N columns and M rows is defined, wherein Aᵢⱼ (the i^{th} row of the j^{th} column of the matrix) indicates the allele state (‘1’ or ‘2’) of the j^{th} SNP for the i^{th} haplotype. The algorithm can eliminate columns of A while preserving as much of the information in P as possible. Quantifying the information in P can be defined using the Shannon Entropy equation:

$$H = - {\sum }_{i = 1}^{M} {P}_{i} ln ( {P}_{i} )$$

According to various embodiments, it can be useful to use an algorithm in lossless mode. According to such embodiments, it can be irrelevant which information measure is used for the haplotype distribution. The algorithm can use other measures of information.

According to various embodiments, the algorithm can consist of two phases (phases I and II). These phases can be performed sequentially. The operations can be outlined in lossless mode as follows below.

In the first phase (Phase I), any column that is identical to another column, or is the exact opposite of another column, can be eliminated. A column in a matrix that is identical to another column can represent a SNP that behaves identically to another SNP for all tested samples. Thus, when the number of DNA samples is large enough to infer the major haplotypes, the redundant SNP will not provide any additional information. Similarly, when a column is the exact opposite of another column in the matrix, this represents a SNP where the behavior can always be predicted from the behavior of another SNP simply by inverting it. Therefore, according to such embodiments, this SNP will not provide new information. According to various embodiments, after phase I, it can be assumed that N columns of matrix A have been reduced to N′ unique columns where N′≦N.

According to various embodiments, in the second phase (Phase II) any column whose elimination does not reduce the number of unique rows, can be eliminated. Each row in a matrix can represent the allelic states of the SNPs for a specific haplotype. Removing a “useful” SNP can eliminate the ability to detect at least one haplotype. According to such an embodiment, two or more haplotypes can register the same allelic state at the remaining SNPs, thereby reducing the number of unique rows. Therefore, if the elimination of a column does not reduce the number of unique rows, it can be omitted.

Phase I can be a “sub-set” of phase II, in the sense that if phase I is skipped, phase II can eliminate the SNPs that phase I would have eliminated. Phase I can be computationally easier to perform than phase II, for example, in lossy mode. Therefore it can be more efficient to begin with phase I.

### EXAMPLE 1

Example 1 illustrates a method according to various embodiments, in lossless mode. Four SNPs and four haplotypes that yield the following allelic responses are illustrated in Table 1.

TABLE 1
Haplotype/SNP Allele State Matrix

| | SNP₁ | SNP₂ | SNP₃ | SNP₄ |
|---|---|---|---|---|
| Haplotype 1 | 1 | 1 | 1 | 2 |
| Haplotype 2 | 2 | 2 | 1 | 1 |
| Haplotype 3 | 2 | 2 | 2 | 1 |
| Haplotype 4 | 1 | 2 | 2 | 2 |

The fourth column is the exact opposite of the first column. This implies that either SNP₄ or SNP₁ is redundant. If SNP₄ is removed from the SNP set, no information is lost. When SNP₁ registers allele “1”, the state of SNP₄ is known as allele “2”, and conversely, when SNP₁ registers allele “2”, the state of SNP₄ is known as allele “1”. Removing SNP₄ leaves the matrix seen in Table 2.

TABLE 2
Haplotype/SNP Allele State Matrix after Phase I

| | SNP₁ | SNP₂ | SNP₃ |
|---|---|---|---|
| Haplotype 1 | 1 | 1 | 1 |
| Haplotype 2 | 2 | 2 | 1 |
| Haplotype 3 | 2 | 2 | 2 |
| Haplotype 4 | 1 | 2 | 2 |

The three columns are unique (including accounting for opposites), thus phase I is complete. N=4 has been reduced to N′=3, as phase II is entered.

Table 3 depicts the three remaining matrices, following the removal of SNP₁, SNP₂, or SNP₃, respectively. The first and the third matrices only have three unique rows, whereas the second matrix has four unique rows. Thus, if the haplotype list is exhaustive, SNP₂ can be eliminated with no loss of haplotype detection.

TABLE 3
Three Possible Haplotype/SNP Allele State Matrices

| | SNP₂ | SNP₃ | SNP₁ | SNP₃ | SNP₁ | SNP₂ |
|---|---|---|---|---|---|---|
| Haplotype 1 | 1 | 1 | 1 | 1 | 1 | 1 |
| Haplotype 2 | 2 | 1 | 2 | 1 | 2 | 2 |
| Haplotype 3 | 2 | 2 | 2 | 2 | 2 | 2 |
| Haplotype 4 | 2 | 2 | 1 | 2 | 1 | 2 |

According to various embodiments, the set {SNP₁, SNP₃} can provide the same haplotype detection ability as the full set {SNP₁, SNP₂, SNP₃, SNP₄}. In Example 1, each phase can cause the elimination of exactly one SNP. However, according to various embodiments, each phase can result in the elimination of multiple SNPs or no SNPs.

### EXAMPLE 2

Further elimination of SNPs, beyond the lossless elimination shown in Example 1 above, can be implemented. According to such embodiments, the retained SNP set can be optimized to minimize the loss of haplotype detection. Phase I can remain unchanged and phase II can select the optimal SNPs to eliminate.

According to various embodiments, for the

$$( \begin{matrix}{N}^{′} \\ k\end{matrix} )$$

possible selections of k SNPs, the entropy H for the resulting P is computed. The selection with the highest H can be chosen as the best selection. When only k out of N′ SNPs are selected, N′−k columns can be eliminated. The resulting matrix (with k columns) can have fewer unique rows than the full matrix (with AN columns). Several “minor” haplotypes can be measured as a single “major” haplotype when a row is repeated more than once. This can occur because with fewer SNPs the ability to make the finer distinction between them is lost. According to various embodiments, the relative frequency (probability) of a “major” haplotype is equal to the sum of the frequencies of the “minor” haplotypes. According to such embodiments, when elimination of columns results in repeating rows, the repeating rows can be combined into a single row, and their respective probabilities can be summed to form a new probability. The vector P can be shorter and can have larger numbers. This can reduce the value of the entropy, H. According to various embodiments, the combination with the smallest reduction of entropy can be deemed the optimal selection. According to various embodiments, if all the rows are unique after elimination of N′−k columns, the entropy is not reduced, and k SNPs can be used with no loss of information, as in Example 1.

Example 2 uses an LD block that was discovered using the Caucasian population panel, in Chromosome 6, overlapping the Human gene TTK (RefSeq ID NM_{—}003318, Celera ID hCG401205) in lossy mode. The block consists of 17 SNPs, and the EM algorithm inferred 8 haplotypes, with two major ones: haplotype 2 and haplotype 7 with frequencies of approximately 43% and 33%, respectively. The remaining 24% of the diversity is spread among the remaining 6 haplotypes. Table 4 summarizes the allelic states of the 17 SNPs, as well as the respective probability, for each of the 8 haplotypes.

TABLE 4
Original Haplotype/SNP Allele State Matrix

| Haplotype | | SNP No. | | | | | | | | | | | | | | | | |
|---|---|---|---|---|---|---|---|---|---|---|---|---|---|---|---|---|---|---|
| Number | P | 1 | 2 | 3 | 4 | 5 | 6 | 7 | 8 | 9 | 10 | 11 | 12 | 13 | 14 | 15 | 16 | 17 |
| 1 | 0.1136 | 1 | 1 | 1 | 1 | 1 | 2 | 1 | 2 | 1 | 2 | 1 | 1 | 2 | 2 | 2 | 1 | 2 |
| 2 | 0.4318 | 1 | 1 | 1 | 1 | 1 | 2 | 1 | 2 | 1 | 2 | 1 | 1 | 2 | 2 | 2 | 2 | 2 |
| 3 | 0.0114 | 1 | 1 | 1 | 2 | 2 | 1 | 2 | 1 | 2 | 1 | 2 | 2 | 1 | 1 | 1 | 1 | 1 |
| 4 | 0.0454 | 1 | 2 | 2 | 1 | 1 | 2 | 1 | 2 | 1 | 2 | 1 | 1 | 2 | 2 | 2 | 1 | 1 |
| 5 | 0.0454 | 2 | 1 | 1 | 1 | 1 | 2 | 1 | 2 | 1 | 2 | 1 | 1 | 2 | 2 | 2 | 1 | 2 |
| 6 | 0.0118 | 2 | 2 | 2 | 2 | 2 | 1 | 2 | 1 | 2 | 1 | 2 | 1 | 1 | 1 | 1 | 1 | 1 |
| 7 | 0.3292 | 2 | 2 | 2 | 2 | 2 | 1 | 2 | 1 | 2 | 1 | 2 | 2 | 1 | 1 | 1 | 1 | 1 |
| 8 | 0.0114 | 2 | 2 | 2 | 2 | 2 | 1 | 2 | 1 | 2 | 2 | 1 | 2 | 1 | 1 | 1 | 1 | 1 |

After running phase I of the algorithm, the number of SNPs is reduced almost immediately to 7, with the remaining SNP set being {SNP₁, SNP₂, SNP₄, SNP₁₀, SNP₁₂, SNP₁₆, SNP₁₇}. All the haplotype information is preserved, including haplotype distribution, as shown in Table 5. The entropy of the original distribution of haplotypes is H(P)=2.0351 bits. Phase II of the algorithm was then performed.

TABLE 5
Haplotype/SNP Allele State Matrix After Phase I

| Haplotype | | | | | | | | |
|---|---|---|---|---|---|---|---|---|
| Number | P | SNP₁ | SNP₂ | SNP₄ | SNP₁₀ | SNP₁₂ | SNP₁₆ | SNP₁₇ |
| 1 | 0.1136 | 1 | 1 | 1 | 2 | 1 | 1 | 2 |
| 2 | 0.4318 | 1 | 1 | 1 | 2 | 1 | 2 | 2 |
| 3 | 0.0114 | 1 | 1 | 2 | 1 | 2 | 1 | 1 |
| 4 | 0.0454 | 1 | 2 | 1 | 2 | 1 | 1 | 1 |
| 5 | 0.0454 | 2 | 1 | 1 | 2 | 1 | 1 | 2 |
| 6 | 0.0118 | 2 | 2 | 2 | 1 | 1 | 1 | 1 |
| 7 | 0.3292 | 2 | 2 | 2 | 1 | 2 | 1 | 1 |
| 8 | 0.0114 | 2 | 2 | 2 | 2 | 2 | 1 | 1 |

Table 6 shows the optimum SNP subset for k SNPs out of the 8 SNPs that survived phase I. Note that the haplotype information is fully preserved all the way down to k=5, therefore if a lossless version of Phase II were run, the minimum SNP size with no loss of information would be 5. At a SNP subset size of 4, some information is lost. In fact, haplotype 8, the rarest haplotype, was merged into haplotype 7, and the ability to distinguish between the two is lost. As a result, 3.5% of the entropy of the resultant haplotype distribution is lost. The optimal selection of 3 SNPs cause haplotypes 3 and 4 to merge and cause haplotypes 6, 7, and 8 to merge, with a total loss of 9.2% of original entropy. The optimal single SNP is SNP₁₆. With single SNP₁₆, the detection ability is reduced to: “haplotype2” or “other.” Since haplotype 2 is the most common, with 43.2% of the frequency, if only a single SNP was chosen, SNP₁₆ would be the most useful choice.

TABLE 6
Lossy Min. SNP Set Example

| No. of SNPs(k) | $\begin{matrix}No . of \\ Combinations \\ ( \begin{matrix}8 \\ k\end{matrix} )\end{matrix}$ | Optimal Set of k SNPs | Haplotype Distribution Resulting from the Optimal SNP Set | Resulting Entropy (H) (bits) |
|---|---|---|---|---|
| 7 | 8 | {SNP₁, SNP₂, | (0.114, 0.432, | 2.0331 |
| | | SNP₄, SNP₁₀, | 0.011, 0.045, 0.045, | |
| | | SNP₁₂, SNP₁₆, | 0.012, 0.329, 0.011) | |
| | | SNP₁₇} | | |
| 6 | 28 | {SNP₁, SNP₄, | (0.114, 0.432, | 2.0351 |
| | | SNP₁₀, SNP₁₂, | 0.011, 0.045, 0.045, | |
| | | SNP₁₆, SNP₁₇} | 0.012, 0.329, 0.011) | |
| 5 | 56 | {SNP₁, SNP₁₀, | (0.114, 0.432, | 2.0351 |
| | | SNP₁₂, SNP₁₆, | 0.011, 0.045, 0.045, | |
| | | SNP₁₇} | 0.012, 0.329, 0.011) | |
| 4 | 70 | {SNP₁, SNP₁₂, | (0.114, 0.432, | 1.9631 |
| | | SNP₁₆, SNP₁₇} | 0.011, 0.045, 0.045, | |
| | | | 0.012, 0.341)) | |
| 3 | 56 | {SNP₁SNP₁₆, | (0.114, 0.432, | 1.8475 |
| | | SNP₁₇} | 0.057, 0.045, 0.352) | |

To validate and assess the utility of the algorithm, genotyping data was used from 11,160 SNPs distributed in a gene-centric fashion across chromosomes 6, 21, and 22, with intragenic spacing averaging 12 Kb, 8 Kb, and 9 Kb, respectively. The SNPs were scored with 5′nuclease assays including TAQMAN-MGB probes from Applied Biosystems' Assays-on-Demand™ SNP Genotyping Products (Foster City, Calif., USA). The samples typed included 45 African-American and 45 Caucasian DNAs from the Coriell Human Diversity Collection available from Coriell Institute for Medical Research, Camden, N.J., USA. LD blocks and haplotypes were computed independently for each population using methods described in Abecasis, et al., Merlin—rapid analysis of dense genetic maps using sparse gene flow trees. Nat Genet 30:97-101 (2002) and Gabriel et al., The structure of haplotype blocks in the human genome Science 296:2225-2229 (2002), both of which are herein incorporated in their entireties by reference. Only blocks of 3 or more SNPs were considered. Therefore, only 4,864 SNPs were used for the African-American population and 7,347 SNPs were used for the Caucasian population. The Caucasian population is known, in general, to have more and longer LD blocks. The algorithm was implemented in MATLAB v 6.1, available from The MathWorks Inc., Natick, Mass., USA, without further optimization. The computations were completed on a 700 MHz PC in less than 1 minute.

Table 5 summarizes the results after applying the algorithm to the haplotype blocks detected in data for chromosomes 6, 21, and 22. The African-American population panel is denoted by ‘A’ and the Caucasian population panel is denoted by ‘C’.

TABLE 7
Results Summary

| | | | Mean | Mean | | Mean | Mean | Mean Min. | |
|---|---|---|---|---|---|---|---|---|---|
| | | Total | Spacing | Spacing | No. of | Block | SNPs | SNP per Block | |
| | | No. of | Between | Between SNPs | Haplotype | Size | per | | <10% |
| Chr. | Pop. | SNPs | SNPs (bp) | in Genes (bp) | Blocks | (bp) | Block | Lossless | Loss |
| 6 | A | 2,504 | 24,386 | 10,840 | 646 | 23,000 | 3.88 | 2.94 | 2.44 |
| | C | 4,009 | 23,694 | 10,630 | 883 | 34,000 | 4.54 | 2.86 | 2.33 |
| 21 | A | 955 | 12,424 | 7,382 | 242 | 14,933 | 3.95 | 2.92 | 2.39 |
| | C | 1,555 | 11,921 | 7,031 | 336 | 21,032 | 4.63 | 2.88 | 2.32 |
| 22 | A | 1,405 | 10,041 | 6,035 | 350 | 13,714 | 4.01 | 2.99 | 2.47 |
| | C | 1,783 | 9,080 | 7,760 | 417 | 17,505 | 4.28 | 2.81 | 2.27 |

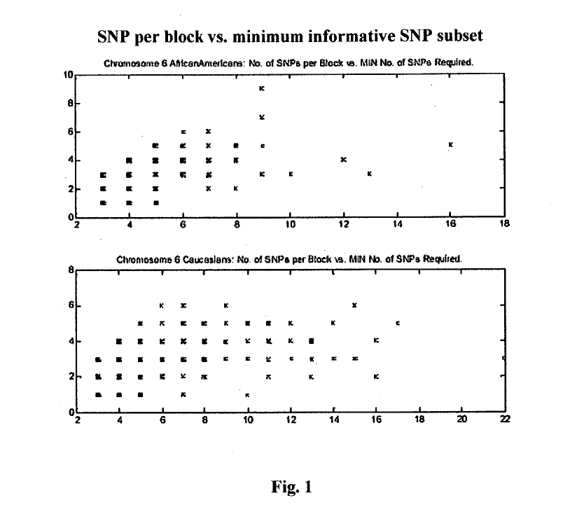

FIG. 1 illustrates the relationship between the original number of SNPs in an LD block (horizontal axis) and the minimum number of SNPs required to genotype the LD block with no loss of information (vertical axis). The thickness of the ‘x’ corresponds to the number of different blocks found in chromosome 6 with the same properties.

Previous algorithms for finding the minimum SNP sub-set have been concerned with complete gene or randomly selected loci, as opposed to LD blocks. In such previous efforts, the number of haplotypes, and more importantly, the amount of information in the haplotype distribution, was expected to be much higher than those presented in the above examples. As a result, these previous efforts were challenged by the numerical complexity, and thus locally optimal solutions were sought. In contrast, algorithms according to various ones of the present embodiments can compute the global optimum, whether in lossless or lossy mode, making it superior to previous efforts.

The method described by Judson et al., How many SNPs does a genome-wide haplotype map require? Pharmacogenomics 3:379 (2002) is essentially equivalent to phase II of the lossy version of various embodiments except that the algorithm is limited to k□11. This is expected, because without the efficient pruning of SNPs performed by phase I, the exponential nature of phase II can result in practically infinite execution time. For example, the largest block found in the above examples consisted of 22 SNPs. In previous efforts, comparing sub-sets of 1 to 2 out of 22 SNPs required examining over 2.4 million combinations. Even after that computation, the optimal solution is not assured since it is only a local optimum. In general, sub-sets of 12 to 22 would also need to be examined in order to assure a global optimum, bringing the total number of combinations to almost 4.2 million. Various embodiments can use phase I to quickly reduce the 22 SNPs to a subset of 4 SNPs. As a result, phase II can find the global optimum (3 SNPs in lossless mode or 2 SNPs with less than 10% loss) in 15 comparisons only.

The method described in the on-line supplement to the paper by Johnson et al., Haplotype tagging for the identification of common disease genes Nat Genet 29:233-237 (2001) compromises the maximization of the information detected by the SNP set with other considerations, e.g. maximization of the individual SNPs' properties. There is little explanation of the method details. One example provided tries to show that a haplotype matrix with full rank cannot be pruned. Note, however, that the haplotype matrix used in Example 1 and shown in Table 1 herein is of full rank, yet was pruned with no loss of information. A counter-example is the 2 by 2 identity matrix, which is know to be full rank, but has the second column as the perfect inverse of the first column, thus providing no new information. The Johnson et al. on-line supplement also provides executable programs, but the maximum subset size is set to k□5, thereby guaranteeing suboptimal results based on the finding that the global optimum is greater than 5 in some blocks.

Previous reports have suggested that the Caucasian population has longer LD blocks than the African-American population, which is considered more diverse. Examples 1 and 2 herein used the same SNPs for both populations, but kept only SNPs which formed LD blocks of 3 or more SNPs. This left more SNPs for the Caucasian population. As Table 5 shows, the Caucasian population yielded more blocks, and more SNPs per block. However, after “compression” of the SNP sets of each block into the minimum required to represent the information (with no loss), the two populations are almost the same. This can reflect the arbitrary criteria that defines an LD block, and can reflect that the criteria was applied uniformly to both populations.

The premise of the algorithm, according to various embodiments, is that the DNA sample size for each population is large enough so that the inferred haplotypes adequately represent reality. There can be a risk that a SNP whose behavior is identical to another SNP (and thus deemed worthless in terms of new information) for the sample size used, could differentiate an additional haplotype inferred with a larger sample size. This risk can be lower for common haplotypes. Rare haplotypes can harbor a causative mutation and can be present in higher frequency in some cases. Experimental errors can eliminate data points and thus can render suboptimal the minimum SNP subset. Therefore, additional SNPs can enhance the minimum SNP subset to enhance robustness.

Data was provided from over 11,000 SNPs with an average spacing of 6 to 11 Kb, across all the genes of chromosomes 6, 21, and 22, and typed on DNA samples of 45 unrelated African-Americans and 45 Caucasians from the Coriell Human Diversity Collection. With no loss of information, the number of SNPs required to capture the haplotype block diversity by 25% for the African-American population and 36% for the Caucasian population was reduced. With a maximum loss of 10% of haplotype distribution information, the SNP reduction was 38% and 49%, respectively, for the two populations. All computations were performed in less than 1 minute for the dataset used.

The availability of human genome data, including putative single nucleotide polymorphisms (SNPs), can enable new clinical and research methods. According to various embodiments, databases containing information on SNPs can be used for conducting genetic studies. Putative SNPs can be validated and can be assembled into a standardized SNP marker map or a database containing data sets corresponding to the standardized SNP marker map. SNP information can be easily accessible and standardized assay reagents can be developed, validated, and made available to enable high throughput and automation, for example, to screen many SNPs on many individuals.

According to various embodiments, a reference SNP database can be produced from SNP and genomic information from both proprietary and public databases. The database can be used for linkage disequilibrium (LD) mapping and can be used to provide, for example, validated, ready-to-use assays and reagents. The database can provide high-density coverage of any known gene regions and can enable easier and more affordable candidate gene association studies and candidate region association studies. Researchers can select SNPs across candidate genes or chromosomal regions that are most suitable for a given study, and can quickly translate that information into practice by, for example, directly obtaining assay protocols and reagents for those SNPs. A “core” set of SNPs and the associated assay reagents can be compiled into a database or library and expanded or refined as additional information become available, such as haplotype definition for some or all of the genome. The SNP database can also be used to compile a fixed set of chromosome-based assays for cost-efficient whole genome association (WGA) studies using, for example, oligonucleotide ligation assay (OLA) PCR Bead Array systems for ultra-high throughput genotyping.

Linkage disequilibrium (LD) is the non-random association of alleles in a chromosomal segment, and can be the basis of all genetic mapping. Selecting SNPs as genetic markers for LD studies involve considering all genetic and assay-specific technical factors that affect the ability to find association between a marker and the susceptibility mutations being mapped.

According to various embodiments, the extent of LD across a genomic region can dictate the SNP density necessary to ensure association between a marker and the allele sought. Early attempts to model the extent of LD predicted very short LD of only a few kilobases (kb). However, recent empirical surveys report average LD levels between 5 kb and 60 kb, and extending up to hundreds of kb, which implies that the number of SNPs required for WGA studies could range from 50,000 to 250,000, and that markers spaced by tens of kb will suffice for candidate gene studies. Common SNPs are the most likely to be useful for LD studies across more than one population since they represent ancient mutations that arose before ethnic group segregation. Simulation studies suggest that common SNPs are more likely than coding SNPs (cSNPs) to be in LD with a given causative allele regardless of whether the allele is present at low or high frequency.

According to various embodiments, common SNPs can be used to assemble a database in a hybrid gene-based approach. SNPs can be considered “common” when the minor allele frequency is, for example, less than 15% in at least one of the populations used for validation. A gene list can include 25,083 gene regions derived by Celera Genomics. A gene region can be defined as bounded by the first and last transcribed base, including untranslated regions, plus 10 kilobases (kb) upstream and downstream to account, for example, for uncharacterized exons and regulatory regions. SNPs can be selected within gene regions at an average density of, for example, one SNP per 10 kb, such that the map can resemble a gene-focused picket fence. Density for specific regions can be adjusted as data on recombination and LD extent emerges. Additional SNPs in intergenic regions, such as, for example, non-coding regions of homology between mouse and human, can be added to a database or library.

Over four million unique SNPs have been reported. However, literature reports state that as few as 50% of SNPs randomly selected from public databases are polymorphic and can yield working assays. According to various embodiments, obtaining a validated SNP assay at the end of a process of validating possible SNPs can be enhanced by defining prioritization criteria. One criterion that can be used to validate a possible SNP is evidence of independent discovery of the minor allele. A data set corresponding to a possible SNP can be cross-referenced against data available in public sources. When a data set corresponding to a possible SNP has no equivalent in the public domain, observation of the minor allele in genotypes of two independent donors can be used to validate the SNP. When a data set corresponding to a possible SNP has a minor allele that is found in only one donor, an independent instance of the minor allele found by searching the consensus assembly of the public Human Genome Project can be used to validate the SNP. A SNP database obtained using the above criteria can include about one million data sets corresponding to SNPs.

According to various embodiments, methods of confirming the existence of a SNP are provided. One step can include identifying a location corresponding to a possible SNP in a polynucleotide in a first collection of data sets. The first collection can contain information on genomic deoxyribonucleic acid (DNA) samples in the form of data sets corresponding to polynucleotides. Another step can include confirming the existence of the SNP if at least one of a number of conditions is present or met. A condition can be that a second collection of data sets containing information on genomic DNA samples contains information that identifies the location as containing the possible SNP. A condition can be that at least two data sets from the first collection of data sets contain information corresponding to a minor allele of the possible SNP at the location. According to various embodiments, the at least two data sets representing genomic DNA samples are obtained from two independent sources. A condition can be that a data set that corresponds to a consensus sequence of genomic DNA samples in a third collection of data sets has the minor allele of the possible SNP. According to various embodiments, the source of the genomic DNA of the consensus sequence and the sources of the genomic deoxyribonucleic acid (DNA) samples from the first collection of data sets are independent. The third database of genomic DNA samples can be, for example, a public database of the Human Genome Project. The first database of at least one genomic DNA sample can be, for example, a proprietary database of the Human Genome Project.

According to various embodiments, a multi-step, high-throughput assay design pipeline can be provided to ensure optimum performance of assays. The methods provided can enable automation, minimize assay failure, and ensure compatibility of the SNP sequence with, for example, TAQMAN probe-based 5′ nuclease chemistry available from Applied Biosystems, Foster City, Calif., and/or other assay formats. A stringent scoring system can be used to select only those SNP context sequences with the highest probability of success.

According to various embodiments, a bioinformatics process can be used to design assays. A step can involve masking SNPs adjacent to the target polymorphism and/or any sequence discrepancy between the Celera and the HGP human genome assembly, within the 600 bases of a context sequence. This can prevent primers and probes from being placed on top of other SNPs and can maximize the chance that the probes will hybridize to the correct genomic sequence.

TAQMAN 5′ nuclease primers and probes can be designed using, for example, the ASSAYS-BY-DESIGN custom oligonucleotide reagent service (Applied Biosystems, Foster City, Calif.). Oligonucleotides can be designed in batch mode without manual intervention, and a scoring scheme can select the best sequences for a given SNP. The design algorithm can implement thermodynamic and heuristic rules and additional empirically-derived factors can increase manufacturability and assay performance. According to various embodiments, probes can be designed successfully for, for example, 97% of SNPs. After this step, a further computational quality-control step can also be performed in the context of the genome that can allow the elimination of potentially problematic SNP targets that may arise from repeated genomic regions, pseudo-SNPs, and/or other possible assembly artifacts.

Finally, the primers and probes can be synthesized, and additional quality-control steps can occur. For example, oligonucleotide integrity can be tested. For further example, assay performance can be tested against a panel of 10 DNA samples. According to various embodiments, assays that pass post-manufacturing quality control can be validated in the population panels.

Assay validation in population panels can ensure that the locus is polymorphic and that the allele frequency is adequate for association studies in a variety of populations. For example, ninety (90) samples from the Coriell Human Variation Collection were obtained. By obtaining individual genotypes from a panel of 45 African Americans, a panel of 45 Caucasians, and a chimp DNA sample (to provide insight into ancestral alleles), sufficient information was obtained to estimate linkage disequilibrium between the SNPs in the LD map and to computationally infer common haplotypes. Assay validation in population panels can provide additional information on the usefulness of the markers, the coverage provided for a given study, and/or provide an independent assessment of assay performance.

According to various embodiments, the performance of each assay that can be comprised of at least one polynucleotide can be benchmarked against criteria, such as, for example: background signal (e.g., low signal in the control experiments run without template); signal generation (e.g., good separation between control experiments run without template and allele clusters); and specificity. A criterion can be that a maximum of three clusters of fluorescing sample and a minimum of two clusters of fluorescing sample must be observed. Another criterion can be that at least 90% of samples yield callable genotypes.

According to various embodiments, a method is provided for compiling a library of polynucleotide data sets that each correspond to polynucleotides that can function as (A) a primer for producing a nucleic acid sequence that is complementary to at least one target nucleic acid sequence including a target SNP, (B) a probe for rendering detectable the at least one target nucleic acid sequence including a target SNP, or (C) both (A) and (B). The method can include the step of selecting for the library polynucleotide data sets that each correspond to a respective polynucleotide that contains a sequence that is complementary to a respective first allele included in each of the at least one target nucleic acid sequences, if, under a set of reaction conditions, a number of parameters are met by each polynucleotide corresponding to the data sets included in the library. These parameters can include: (1) the respective polynucleotide has a background signal value less than or equal to a first defined value, where the background signal value is a first normalized ratio of a fluorescence intensity of the respective polynucleotide reacted with first assay reactants in the absence of the target nucleic acid sequence, and under first conditions of fluorescence excitation, to a dye fluorescence intensity of a passive-reference dye under the first conditions; (2) the respective polynucleotide has a signal generation value of greater than or equal to a second defined value, wherein the signal generation value is the difference between (i) a second normalized ratio of the fluorescence intensity of the respective polynucleotide reacted with the first assay reactants in the presence of the target nucleic acid sequence, to the dye fluorescence intensity and (ii) the background signal value; (3) the respective polynucleotide has a specificity value of less than or equal to a third defined value, wherein the specificity value is the difference between (i) a third normalized ratio of the fluorescence intensity of the respective polynucleotide reacted with second assay reactants that contain a second allele included in the at least one target nucleic acid sequence to the dye fluorescence intensity, wherein the second allele differs from the first allele, and (ii) the background signal value; (4) at least one individual from a population of individuals has a genotype identifiable under the first conditions, that results from reacting the respective polynucleotide with the first assay reactants and in the presence of the target nucleic acid sequence, wherein the population includes at least one individual that has the identifiable genotype and at least one individual that does not have the identifiable genotype; and (5) at least one individual from the population has an identifiable minor allele of the identifiable genotype, under the first conditions that results from reacting the respective polynucleotide with the first assay reactants in the presence of the target nucleic acid sequence, wherein the population includes at least one individual that has the identifiable minor allele, and at least one individual that does not have the identifiable minor allele. Various embodiments of these and/or other parameters can be used in deciding whether to select a polynucleotide or a polynucleotide data set for a library.

According to various embodiments, the first reaction conditions can comprise a 900 nM final primer concentration and a 250 nM final probe concentration under thermal cycling conditions. The first defined value can be about 2.0, the second defined value can be about 1.0, and the third defined value can be about 2.0. At least, for example, 0.01% of individuals from the population can have the identifiable genotype. At least 10% of individuals from the population can have the identifiable genotype. At least 20% of individuals from the population can have the identifiable genotype. The identifiable genotype can result from reacting the respective polynucleotide with the first assay reactants in the presence of the target nucleic acid sequence. The reaction can occur under the first conditions. The population can have a frequency of the minor allele of greater than or equal to about 5%. For example, the minor allele frequency can be greater than or equal to about 10%. The minor allele frequency can be greater than or equal to about 15%. According to various embodiments, methods can include not selecting a second polynucleotide data set that corresponds to a second polynucleotide if one or more of parameters (1) - (5), above, is not met by the second polynucleotide.

A library of polynucleotide data sets can be compiled using methods according to various embodiments. A library of assays can be compiled using methods according to various embodiments. The method can include manufacturing a library of assays wherein each assay can be made using a polynucleotide data set compiled in the library. A library of polynucleotides can be compiled by manufacturing polynucleotides corresponding to polynucleotide data sets compiled using methods according to various embodiments. A library of assays can be compiled using methods according to various embodiments.

According to various embodiments, a method of detecting a SNP can be provided. A step of the method can be reacting a sample containing a target nucleic acid sequence that has a target SNP with an assay selected from the library of assays compiled according methods described herein. A step can be determining the genotype of the target nucleic acid sequence that has the target SNP by detecting a characteristic attributable to the genotype of the target SNP in the sample.

According to various embodiments, a method is provided for compiling a library of polynucleotide data sets that correspond to polynucleotides that each can function as (A) a primer for producing a nucleic acid sequence that is complementary to at least one target nucleic acid sequence including a target SNP, (B) a probe for rendering detectable the at least one target nucleic acid sequence including a target SNP, or (C) both (A) and (B). The method can include the step of determining a background signal value by calculating a first normalized ratio of a fluorescence intensity of a respective polynucleotide that contains a sequence that is complementary to a first allele included in the at least one target nucleic acid sequence, reacted with first assay reactants in the absence of the target nucleic acid sequence, and under first conditions of fluorescence excitation, to a dye fluorescence intensity of a passive-reference dye under the first conditions. The method can include the step of comparing a difference between (i) a second normalized ratio of the fluorescence intensity of the respective polynucleotide reacted with the first assay reactants in the presence of the target nucleic acid sequence, to the dye fluorescence intensity, and (ii) the background signal value. The method can include the step of comparing a difference between (i) a third normalized ratio of the fluorescence intensity of the respective polynucleotide reacted with second assay reactants that contain a second allele included in the at least one target nucleic acid sequence to the dye fluorescence intensity, wherein the second allele differs from the first allele, and (ii) the background signal value. The method can include the step of determining whether at least one individual from a population of individuals has a genotype identifiable under the first conditions that results from reacting the respective polynucleotide with the first assay reactants and in the presence of the target nucleic acid sequence, wherein the population includes at least one individual that has the identifiable genotype and at least one individual that does not have the identifiable genotype. The method can include the step of determining whether at least one individual from the population has an identifiable minor allele of the identifiable genotype, under the first conditions, that results from reacting the respective polynucleotide with the first assay reactants in the presence of the target nucleic acid sequence. The method can include a combination of some of or all of these steps.

According to various embodiments, the polynucleotide data set corresponding to the respective polynucleotide can be selected for the library if, for example, the background signal value in parameter (1) is less than or equal to about two, if the ratio from the comparison in parameter (2) is greater than or equal to about one, if the ratio from the comparison in parameter (3) is less than or equal to about two, if the at least one individual of parameter (4) has the identifiable genotype, and if the at least one individual of parameter (5) has the identifiable minor allele.

A library of polynucleotide data sets can be compiled using methods according to various embodiments. A library of polynucleotides can be compiled by manufacturing polynucleotides corresponding to polynucleotide data sets compiled using a method or methods according to various embodiments.

A method of compiling a library of assays can be provided. The method can include manufacturing a library of assays, wherein each assay is manufactured using a polynucleotide data set compiled in a library according to various embodiments.

According to various embodiments, methods of detecting a SNP can be provided. The method can include the step of reacting a sample containing a target nucleic acid sequence that has a target SNP with an assay selected from the library of assays compiled using a method or methods according to various embodiments. A step can include determining the genotype of the target nucleic acid sequence that has the target SNP by detecting a characteristic attributable to the genotype of the target SNP in the sample.

### EXAMPLE 3

Using chromosome 22 as a pilot project, 2,260 SNP assays were validated. Of those assays tested, 94% of the SNPs tested with population panels were polymorphic and 90% of the assays passed performance criteria. When a minor allele frequency cut-off of 15% or greater was used, 85% of the samples from the African-American panel, and 90% from the Caucasian panel, met the performance criteria. Ninety-nine percent (99%) of SNPs had a minor allele frequency of 15% or greater in at least one of the population panels. Ninety-six percent (96%) of samples had a minor allele frequency of 20% or greater in at least one population tested, and 62% of samples had a minor allele frequency of 20% or greater in both populations tested.

According to various embodiments, an automatic allele calling software can be used to automatically analyze validated assay data without user intervention. According to various embodiments, at least, for example, 90% of the assay data can be processed automatically to identify an allele. According to various embodiments, an automated validation process can be used for high volume commercial or research purposes.

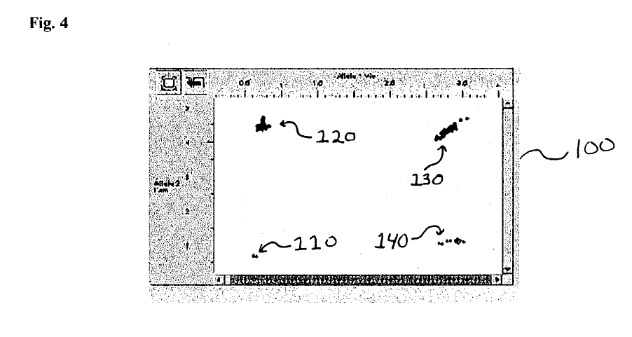

FIG. 4 is a plot 100 of fluorescence data from many SNP assays according to various embodiments. The x-axis represents relative fluorescence of a 6-FAM dye label and the y-axis represents relative fluorescence of a VIC dye label. Cluster 110 represents the relative fluorescence of control samples having probes labeled with 6-FAM and VIC, respectively. The control samples did not contain a target nucleic acid sequence. The background signal value is the average of the relative fluorescence of the control samples as represented by cluster 110 in FIG. 4. The background signal value in FIG. 4 is less than about 2.0. Cluster 120 represents the relative fluorescence of samples having homozygous alleles (allele 2) that hybridized with probes labeled with 6-FAM. Cluster 130 represents the relative fluorescence of samples having heterozygous alleles (alleles 1 and 2) that hybridized with probes labeled with VIC and 6-FAM, respectively. Cluster 140 represents the relative fluorescence of samples having homozygous alleles (allele 1) that hybridized with probes labeled with VIC. The signal generation value for the assays is based, at least in part, on the average of the relative fluorescence of at least one of clusters 120, 130, and 140.

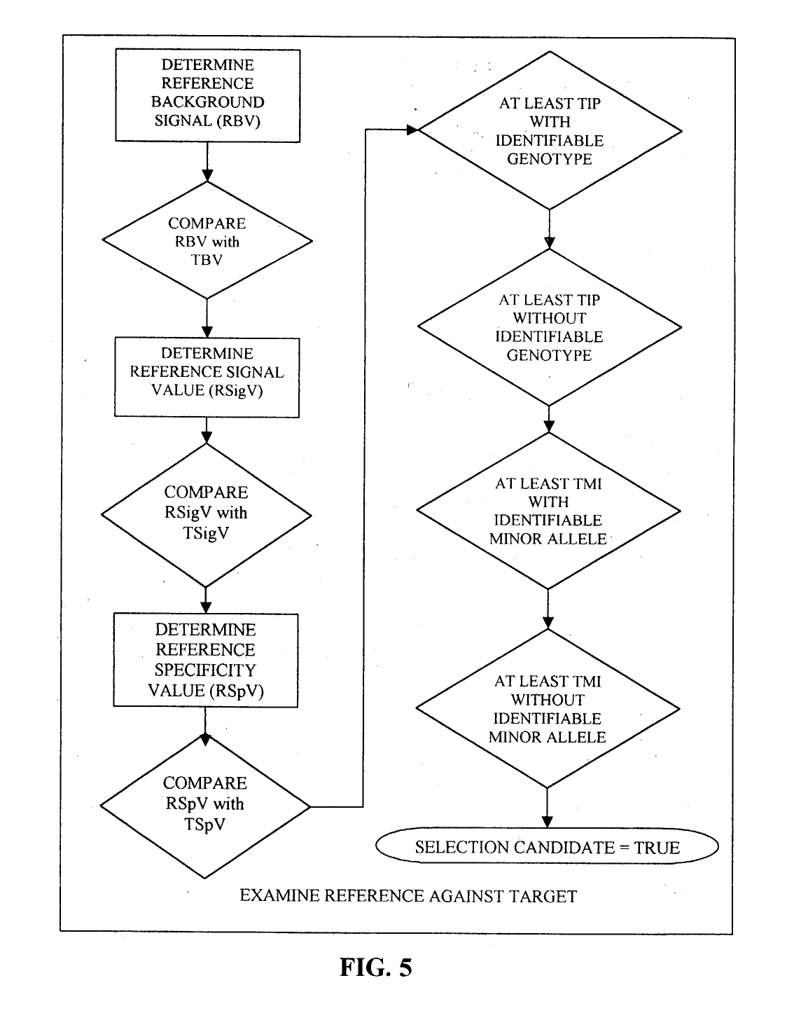

FIG. 5 is a flowchart showing a comparison of reference values determined by experimental assays according to various embodiments against target values. As used herein TBV is a target background value; TsigV is the target signal value; TSpV is the target specificity value; TIP is the target identifiable percentage, or the minimum frequency that the assay produces an identifiable genotype; and TMI is the target minor allele frequency, or the minimum frequency that the minor allele appears in the population.

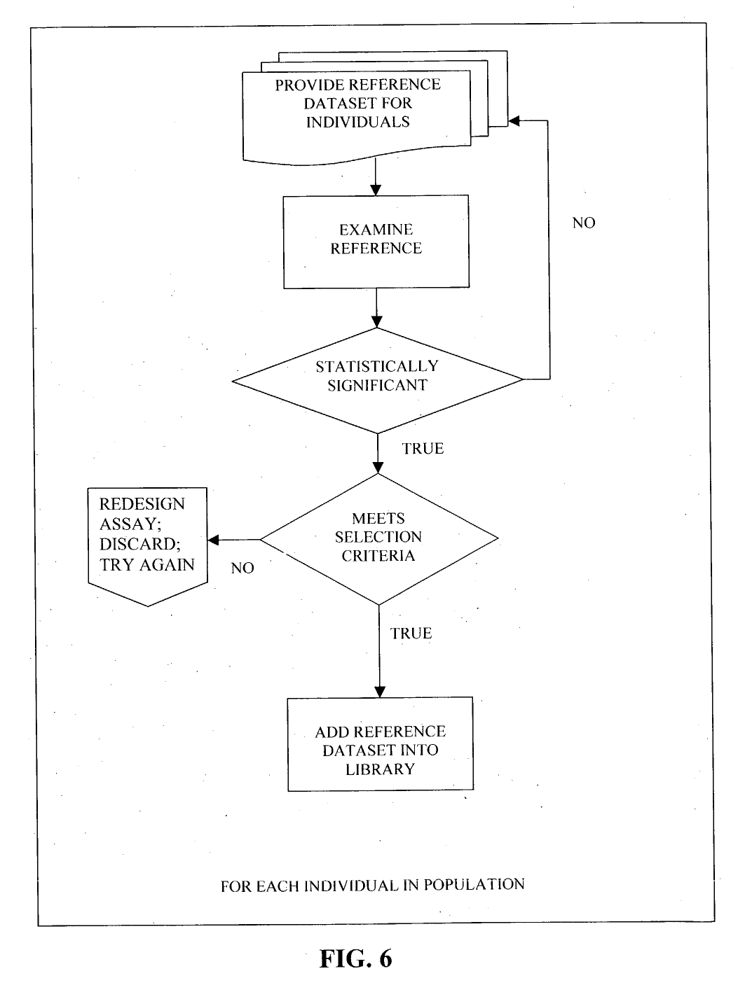

FIG. 6 is a flowchart that illustrates the selection of a data set into a library of data sets, according to various embodiments. Experimental or reference data is obtained by performing an assay against individuals in a population. Experimental data can be obtained at least until results are statistically significant for a given population. When a statistically significant sample size has been determined, the experimental data can be processed according to various embodiments as shown, for example, in FIG. 5. If the selection criteria are met, the data set corresponding to polynucleotides used in the experimental assays can be added into the library. If the selection criteria are not met, the polynucleotide can be redesigned or discarded, where the expendable assays can be repeated.

According to various embodiments, SNP assays and reagents can use easily automated chemistry and can be compatible with readily available high-throughput instrumentation and software systems. According to various embodiments, SNP assays and reagents can have few enzymatic steps, no post-reaction transfer of liquids, and/or universal reaction conditions that can facilitate robotic liquid handling automation. The assays, reagents, and/or high-throughput workflow can be easy to implement and automate and/or can use components that are ready-to-use out-of-the-box and require no optimization.

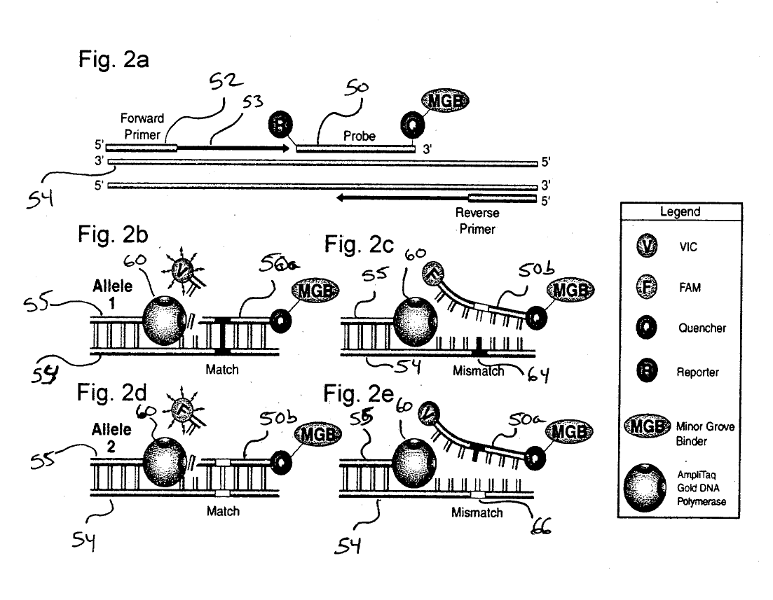

TAQMAN probe-based 5′ nuclease assay chemistry, available from Applied Biosystems, Foster City, Calif., can meet almost any assay requirement and can unite PCR amplification and signal generation into a single step, thereby simplifying automation of both reaction set-up and data collection. In the TAQMAN system, a hybridization probe with fluorogenic and quencher tags is cleaved by the 5′ nuclease activity of thermus aquaticus (Taq) DNA polymerase during PCR amplification. Cleavage produces fluorescence by freeing the fluorogenic molecule from the quencher. By using two probes, one specific to each allele of the SNP and labeled with distinct fluorogenic tags, both alleles can be specifically detected in a single tube. In addition, the fluorescent 5′ nuclease assays can be part of an easy-to-use, automated system for SNP genotyping. FIGS. 2a-2e provide an overview of the TAQMAN probe-based 5′ nuclease assay chemistry for SNP genotyping.

The TAQMAN system is adapted to provide allelic discrimination and high-throughput SNP genotyping. Chemistry improvements have increased assay design flexibility, enabled easy protocol standardization, enabled the use of universal reagents, and reduced background fluorescence, all of which can be desirable for high throughput SNP processing and allelic discrimination.

For example, previous probes of up to 30 bases were required to achieve the specificity required for scoring SNPs. The conjugation of a DNA minor groove binder (MGB) to a probe can significantly stabilize probe-template complexes, enabling the use of probes in the 13-mer to 20-mer size range. Such conjugated probes can have better mismatch discrimination, can be easier to design for challenging genetic regions such as those high in GC content or those in variable context sequences, and/or can increase the signal-to-noise ratio by bringing the quencher closer to the fluorescent tag. For high-throughput SNP scoring, conjugated probes can increase the melting temperature window that can be used in reaction protocols, thereby allowing all the SNP assays to run under identical conditions.

Previous 5′ nuclease quenchers emitted their own fluorescence, therefore signal detection was complicated. New non-fluorescent quenchers can provide improved signal detection and can facilitate automated allele calling, another desirable feature useful for high-throughput SNP scoring.

In a pharmacogenomic study, the most precious reagent can be the DNA sample itself High-quality DNA quantification using real-time analysis on the ABI PRISM 7900HT Sequence Detection System with the TAQMAN RNase P Control Reagents Kit, both available from Applied Biosystems, Foster City, Calif., permits optimal amounts of DNA per reaction to maximize study efficiency. The 7900HT system can use a 5 microliter reaction volume and can consume one nanogram of DNA per genotyping reaction, thereby minimizing reagent costs and conserving DNA template samples.

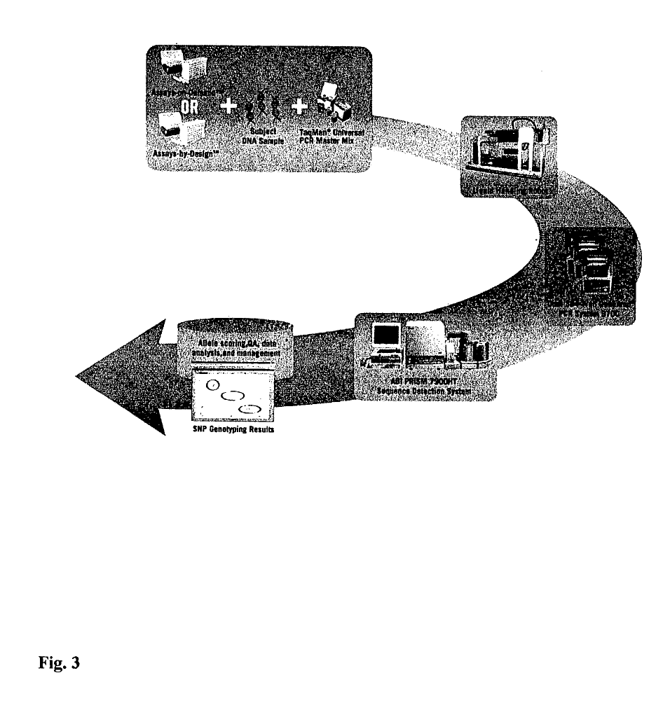

The 5′ nuclease assay can be suited for automation because of its easy, three-step workflow. A universal master mix, including probes and primers, can be added directly to plates of dry or fresh DNA using standard robotics. Plates can be sealed and cycled using standard thermal cyclers, such as, for example, the Applied Biosystems Dual 394-Well GENEAMP PCR System 9700 thermal cycler, available from Applied Biosystems, Foster City, Calif. Following cycling, plates can be automatically read on the 7900HT that can support the collection of more than 250,000 genotypes per day. In addition, the availability of thermal cyclers with automated lid handling can increase throughput by enabling robotics integration for 24-hour unattended operation. Automation software can also increase both quality and throughput. For example, automation of allele calling can remove inter-technician variability, increasing confidence in data quality and reducing the time spent on data analysis by 8.5 person-hours per day. FIG. 3 provides an example of an automated workflow system.

According to various embodiments, an assay that uses two different types of probes can be provided wherein the polynucleotide and the reporter dyes differ. For example, the first type of probe can have a first polynucleotide with a VIC reporter dye attached to the 5′ end of the first polynucleotide, and the second type of probe can have a second polynucleotide with a 6-FAM reporter dye attached to the 5′ end of the second polynucleotide, and the first and second polynucleotides can differ by at least one nucleic acid residue at the same location in the polynucleotide when the polynucleotides are aligned 5′ to 3′. The dye-labeled probes can be adopted to perform a heterozygous assay or a homozygous assay.

The probe can anneal to a complementary sequence between the forward and reverse primer sites. At the time of annealing, the probe is intact and the proximity of the reporter dye to the quencher can result in suppression of fluorescence of the reporter dye. A polymerase can cleave a reporter dye only when the probe has completely, mostly, or substantially hybridized to the target DNA sequence. When the reporter dye is cleaved from the probe, the relative flouorescence of the reporter dye increases. The increase in relative fluorescence can be caused to only occur if the amplified target DNA sequence is complementary, mostly complementary, or substantially complementary to the probe. Therefore, the fluorescent signal generated by PCR amplification can indicate which alleles are present in a sample. Mismatches between a probe and a target DNA sequence can reduce efficiency of probe hybridization and/or a polymerase can be more likely to displace a mismatched probe without cleaving it and therefore not produce a fluorescent signal. For example, if one of two possible reporter dyes fluoresce during an assay, then the presence of a homozygous gene is indicated. For further example, if both possible reporter dyes fluoresce during an assay, then the presence of a heterozgous gene is indicated.

According to various embodiments, at least one primer can be provided, wherein the primer can be a sequence that is shorter than the target DNA sequence. The primer can have a polynucleotide and/or a minor groove binder. The primer can be a sequence that is complimentary to, or mostly complimentary to, the target DNA sequence. The primer can be at least 90% homologous to a corresponding length of the target DNA sequence, at least 80% homologous to a corresponding length of the target DNA sequence, at least 70% homologous to a corresponding length of the target DNA sequence, or at least 50% homologous to a corresponding length of the target DNA sequence.

According to various embodiments, a thermostable DNA polymerase, such as, for example, thermus aquaticus (Taq), and at least 4 embodiments of a deoxyribonucleic acid (e.g., adenosine, tyrosine, cytosine, and guanine) can be provided. The polymerase can be, for example, AMPLITAQ GOLD, available from Applied Biosystems, Foster City, Calif.

According to various embodiments, components of a fluorogenic 5′ nuclease assay or other assay reagents that utilize 5′ nuclease chemistry, for example, TAQMAN minor groove binder probes, available from Applied Biosystems, Foster City, Calif., can be provided. Some or all of the above-listed components can be replaced by or used with commercially-available products, for example, buffers or AMPLITAQ GOLD PCR MASTER MIX (Applied Biosystems, Foster City, Calif.).

According to various embodiments, a high-quality LD map of validated SNPs was created by integrating information from both public and private human genome efforts. A set of over 200,000 validated, easy-to-use, individual SNP assays and TAQMAN ready-to-use assay reagents created by using methods according to various embodiments can be provided. A minor groove binder and a non-fluorescent quencher, and the integration of the 5′ nuclease chemistry with an automated detection system, such as, for example, the 7900HT, can be used. According to various embodiments. A web-based bioinformatics and ordering system can be provided where a customer can search for SNPs and order assay reagents, thus reducing the time and costs associated with candidate-gene and candidate-region association studies. According to various embodiments, and LD map can, for example, enable candidate-gene and candidate-region association studies using 5′ nuclease chemistry and/or be implemented on an ultra-high throughput SNP genotyping platform to enable WGA studies. According to such embodiments, the 5′ nuclease chemistry system can leverage the specificity of the OLA-PCR assay chemistry and the highly parallel detection of, for example, BEADARRAY technology, available from Illumina, Inc., San Diego, Calif. According to such embodiments, the system can enable the generation of about 2,100,000 genotypes per day and all components of the assay can be universal except for, for example, the SNP-specific OLA probes.

According to various embodiments, assays for over 4,000,000 SNPs from the Celera database can cover every gene in the human genome. According to various embodiments, many SNPs can have the necessary variability for genetic association studies and assays for the SNPs can be provided. According to various embodiments, assays can be grouped together into convenient SNP sets optimized for specific assays such as, for example, p450 genotyping and disease-specific gene studies.

FIGS. 2a-2e are schematic diagrams showing the interaction of components that can be part of a mixture of reagents according to various embodiments. In FIG. 2a, primer 52 has annealed to template strand 54. Replication of the template strand from primer 52 will occur in the 5′ to 3′ direction. Probe 50, including a generic reporter dye R, quencher Q, and minor groove binder MGB, has annealed to the template strand 54. Arrow 53 shows that as the complementary strand (not shown) is produced from the template strand 54 starting at the forward primer 52, the complementary strand will meet probe 50. FIG. 2b shows the complementary strand 55 as it meets probe 50a. Polymerase 60 cleaves VIC reporter dye V during the production of complementary strand 55 given that probe 50a has annealed to the target strand 54 because the target strand 54 and the probe 50a are completely complementary. FIG. 2c shows the complementary strand 55 as it meets probe 50b. Polymerase 60 does not cleave FAM reporter dye F during the production of complementary strand 55 given that probe 50b has not hybridized with the target strand 54 because of a mismatched base pair at location 64. FIG. 2d shows the complementary strand 55 as it meets probe 50b. Polymerase 60 cleaves FAM reporter dye F during the production of complementary strand 55 given that probe 50b has annealed to the target strand 54 because the target strand 54 and the probe 50b are completely complementary. FIG. 2e shows the complementary strand 55 as it meets probe 50a. Polymerase 60 does not cleave VIC reporter dye V during the production of complementary strand 55 given that the probe 50a has not hybridized with the target strand 54 because of a mismatched base pair at location 66.

The present invention relates to the foregoing and other embodiments as will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method of compiling a library of polynucleotide data sets that correspond to polynucleotides that each can function as (A) a primer for producing a nucleic acid sequence that is complementary to at least one target nucleic acid sequence including a target Single Nucleotide Polymorphism (SNP), (B) a probe for rendering detectable the at least one target nucleic acid sequence including a target SNP, or (C) both (A) and (B), the method comprising the steps of:
selecting for the library polynucleotide data sets that each correspond to a respective polynucleotide that contains a sequence that is complementary to a respective first allele included in each of the at least one target nucleic acid sequences, if, under a set of reaction conditions: (1) the respective polynucleotide has a background signal value less than or equal to a first defined value, where the background signal value is a first normalized ratio of a fluorescence intensity of the respective polynucleotide reacted with first assay reactants in the absence of the target nucleic acid sequence, and under first conditions of fluorescence excitation, to a dye fluorescence intensity of a passive-reference dye under the first conditions; (2) the respective polynucleotide has a signal generation value of greater than or equal to a second defined value, wherein the signal generation value is the difference between (i) a second normalized ratio of the fluorescence intensity of the respective polynucleotide reacted with the first assay reactants in the presence of the target nucleic acid sequence, to the dye fluorescence intensity and (ii) the background signal value; (3) the respective polynucleotide has a specificity value of less than or equal to a third defined value, wherein the specificity value is the difference between (i) a third normalized ratio of the fluorescence intensity of the respective polynucleotide reacted with second assay reactants that contain a second allele included in the at least one target nucleic acid sequence to the dye fluorescence intensity, wherein the second allele differs from the first allele, and (ii) the background signal value; (4) at least one individual from a population of individuals has a genotype identifiable under the first conditions, that results from reacting the respective polynucleotide with the first assay reactants and in the presence of the target nucleic acid sequence, wherein the population includes at least one individual that has the identifiable genotype and at least one individual that does not have the identifiable genotype; and (5) at least one individual from the population has an identifiable minor allele of the identifiable genotype, under the first conditions that results from reacting the respective polynucleotide with the first assay reactants in the presence of the target nucleic acid sequence, wherein the population includes at least one individual that has the identifiable minor allele, and at least one individual that does not have the identifiable minor allele.

2. The method of claim 1, wherein the reaction conditions comprise a 900 nM final primer concentration and a 250 nM final probe concentration under thermal cycling conditions.

3. The method of claim 1, wherein the first defined value is about 2.0, the second defined value is about 1.0, and the third defined value is about 2.0.

4. The method of claim 1, wherein at least 0.01% of individuals from the population have the identifiable genotype, under the first conditions that results from reacting the respective polynucleotide with the first assay reactants and in the presence of the target nucleic acid sequence, and the population has a frequency of the minor allele of greater than or equal to about 10%.

5. The method of claim 4, wherein at least about 1.0% of individuals from the population have the identifiable genotype.

6. The method of claim 4, wherein at least about 5.0% of individuals from the population have the identifiable genotype.

7. The method of claim 4, wherein at least about 10.0% of individuals from the population have the identifiable genotype.

8. The method of claim 1, wherein the method further includes not selecting a second polynucleotide data set that corresponds to a second polynucleotide if one or more of parameters (1)-(5) is not met by the second polynucleotide.

9. A library of polynucleotide data sets compiled using the method of claim 1.

10. A method of compiling a library of assays, the method comprising manufacturing a library of assays wherein each assay is made using a polynucleotide data set compiled in the library of claim 9.

11. A library of polynucleotides, the library compiled by manufacturing polynucleotides corresponding to polynucleotide data sets compiled using the method of claim 1.

12. A library of assays compiled using the method of claim 10.

13. A method of detecting a SNP, comprising the steps of:
reacting a sample containing a target nucleic acid sequence including a target SNP with an assay selected from the library of assays compiled according to the method of claim 12; and
determining the genotype of the target nucleic acid sequence including the target SNP by detecting a characteristic attributable to the genotype of the target SNP in the sample.

14. A method of compiling a library of polynucleotide data sets that correspond to polynucleotides that each can function as (A) a primer for producing a nucleic acid sequence that is complementary to at least one target nucleic acid sequence including a target SNP, (B) a probe for rendering detectable the at least one target nucleic acid sequence including a target SNP, or (C) both (A) and (B), the method comprising the steps of:
(1) determining a background signal value by calculating a first normalized ratio of a fluorescence intensity of a respective polynucleotide that contains a sequence that is complementary to a first allele included in the at least one target nucleic acid sequence, reacted with first assay reactants in the absence of the target nucleic acid sequence, and under first conditions of fluorescence excitation, to a dye fluorescence intensity of a passive-reference dye under the first conditions;
(2) comparing a difference between (i) a second normalized ratio of the fluorescence intensity of the respective polynucleotide reacted with the first assay reactants in the presence of the target nucleic acid sequence, to the dye fluorescence intensity, and (ii) the background signal value;
(3) comparing a difference between (i) a third normalized ratio of the fluorescence intensity of the respective polynucleotide reacted with second assay reactants that contain a second allele included in the at least one target nucleic acid sequence to the dye fluorescence intensity, wherein the second allele differs from the first allele, and (ii) the background signal value;
(4) determining whether at least one individual from a population of individuals has a genotype identifiable under the first conditions that results from reacting the respective polynucleotide with the first assay reactants and in the presence of the target nucleic acid sequence, wherein the population includes at least one individual that has the identifiable genotype and at least one individual that does not have the identifiable genotype; and
(5) determining whether at least one individual from the population has an identifiable minor allele of the identifiable genotype, under the first conditions that results from reacting the respective polynucleotide with the first assay reactants in the presence of the target nucleic acid sequence.

15. The method of claim 14, wherein a polynucleotide data set corresponding to the respective polynucleotide is selected for the library if the background signal value in parameter (1) is less than or equal to about two, if the ratio from the comparison in parameter (2) is greater than or equal to about one, if the ratio from the comparison in parameter (3) is less than or equal to about two, if the at least one individual of parameter (4) has the identifiable genotype, and if the at least one individual of parameter (5) has the identifiable minor allele.

16. A library of polynucleotide data sets compiled using the method of claim 14.

17. A method of compiling a library of assays, the method comprising manufacturing a library of assays wherein each assay is made using a polynucleotide data set compiled in the library of claim 16.

18. A library of polynucleotides, the library compiled by manufacturing polynucleotides corresponding to polynucleotide data sets compiled using the method of claim 14.

19. A library of assays compiled using the method of claim 17.

20. A method of detecting a SNP, comprising the steps of:
reacting a sample containing a target nucleic acid sequence including a target SNP with an assay selected from the library of assays compiled according to the method of claim 19; and
determining the genotype of the target nucleic acid sequence including the target SNP by detecting a characteristic attributable to the genotype of the target SNP in the sample.

21. A method of confirming the existence of a SNP, the method comprising the steps of:
identifying a location corresponding to a possible SNP in a polynucleotide in a first collection of data sets containing information on genomic deoxyribonucleic acid (DNA) samples in the form of data sets corresponding to polynucleotides; and
confirming the existence of the SNP if at least one of the following conditions is met:
(1) a second collection of data sets containing information on genomic deoxyribonucleic acid (DNA) samples contains information that identifies the location as containing the possible SNP;
(2) at least two data sets from the first collection of data sets contain information corresponding to a minor allele of the possible SNP at the location, wherein the at least two data sets represent genomic deoxyribonucleic acid (DNA) samples obtained from two independent sources; and
(3) a data set that corresponds to a consensus sequence of genomic deoxyribonucleic acid (DNA) samples in a third collection of data sets contains the minor allele of the possible SNP, wherein a source of the consensus sequence of genomic deoxyribonucleic acid (DNA) samples and sources of the genomic deoxyribonucleic acid (DNA) samples from the first collection of data sets are independent.

22. The method of claim 21 wherein the third database of genomic DNA samples is a public database of the Human Genome Project.

23. The method of claim 21 wherein the first database of at least one genomic DNA sample is a proprietary database of the Human Genome Project.

24. A library that contains data corresponding to respective oligonucleotides that can function as assays to detect Single Nucleotide Polymorphisms (SNPs), the library comprising a number of data sets corresponding to not more than a sufficient number of oligonucleotides necessary to provide a collection of assays that provides a maximum statistical loss of haplotype diversity, across a human genome, of less than ten (10) percent.

25. The library of claim 24, wherein loss of haplotype diversity is less than five (5) percent.

26. The library of claim 24, wherein there is no loss of haplotype diversity.

27. The library of claim 24, wherein the haplotype comprises at least one gene.

28. The library of claim 24, wherein the sufficient number of oligonucleotides is obtained by the method of:
(1) providing a matrix comprised of data representing haplotype blocks and SNP locations, wherein the columns contain data representing existence of respective SNPs within a haplotype block and the rows contain data representing respective haplotype blocks; and
(2) eliminating at least one column, wherein elimination of the at least one column does not reduce the number of rows in the matrix that contains non-duplicative information.

29. The library of claim 28, further comprising the step of eliminating at least one column of the matrix that is one of (i) identical to a second column of the matrix, and (ii) completely opposite to a second column of the matrix.

30. A library of assays corresponding to claim 24.

31. The library of claim 24, comprising data sets representing from about 100,000 to about 500,000 oligonucleotides.

32. The library of claim 30, comprising from about 100,000 to about 500,000 assays.

33. The library of claim 24, wherein the sufficient number of oligonucleotides is obtained by:
(1) providing a matrix comprised of data representing haplotype blocks and SNP locations, wherein the columns contain data representing existence of respective SNPs within a haplotype block and the rows contain data representing respective haplotype blocks; and
(2) selecting of a set of SNPs from a certain entropy by eliminating at least one column.

34. The library of claim 31, further comprising the step of eliminating at least one column of the matrix that is one of (i) identical to a second column of the matrix, and (ii) completely opposite to a second column of the matrix.
